(19) United States
(12) Patent Application Publication
(10) Pub. No.: US 2009/0163948 A1
(43) Pub. Date: Jun. 25, 2009

(54) MANIPULATOR SYSTEM AND METHOD OF CONTROLLING MANIPULATOR

(75) Inventors: Takamitsu Sunaoshi, Yokohama-shi (JP); Makoto Jinno, Ashigarakami-gun (JP); Ryohei Katsuki, Fujinomiya-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignee: KABUSHIKI KAISHA TOSHIBA, Minato-ku (JP)

(21) Appl. No.: 12/340,818

(22) Filed: Dec. 22, 2008

(30) Foreign Application Priority Data
Dec. 21, 2007 (JP) 2007-329772

(51) Int. Cl. A61B 17/29

(52) U.S. Cl. 606/205

(57) ABSTRACT

A medical manipulator system includes a manipulator including a working unit having a distal-end working unit, the attitude of which can be changed by a motor, and a controller for controlling the manipulator. When the controller performs an origin return process for moving a gripper of the distal-end working unit to an origin position at an end of an operating range thereof, the controller issues a first control target value to the motor indicative of a virtual position beyond the origin. Thereafter, the controller issues a second control target value to the motor indicative of the origin. The first control target value represents a position, which is over from the origin P0 by a distance greater than a value corresponding to an error ε between a control target position for the gripper and the actual position thereof.

### BACKGROUND OF THE INVENTION

1. Field of the Invention

The present invention relates to a manipulator system including a manipulator having an actuating unit, which can be actuated by an actuator in order to change the attitude thereof, and a controller for controlling the manipulator. The present invention also concerns a method of controlling such a manipulator.

2. Description of the Related Art

According to a laparoscopic surgical operation process, small holes are opened in the abdominal region, for example, of a patient, and an endoscope, forceps or manipulators, etc., are inserted into the holes. A surgeon performs a surgical operation on the patient with the manipulators or forceps while watching an image captured by the endoscope and displayed on a display monitor. Since the laparoscopic surgical operation process does not require a laparotomy to be performed, it is less burdensome on the patient and greatly reduces the number of days that the patient is required to spend before recovering from the operation and being released from the hospital. Thus, the range of surgical operations to which such a laparoscopic surgical process is applicable is expected to increase.

As disclosed in JP 2004-105451 A, for example, a manipulator system comprises a manipulator and a controller for controlling the manipulator. The manipulator comprises a manually operable operating unit and a working unit replaceably mounted on the operating unit.

The working unit (instrument) comprises a long joint shaft and a distal-end working unit (also referred to as an end effector) mounted on the distal end of the joint shaft. The operating unit includes actuators (motors) therein for actuating the working unit through wires. The wires are wound around respective pulleys disposed in a proximal end portion of the working unit. The controller energizes the motors of the operating unit in order to cause the pulleys to move the wires back and forth.

Various different working units, including a gripper, scissors, an electrosurgical knife, an ultrasonic knife, a medical drill, etc., are used to perform respective surgical techniques in a laparoscopic surgical operation process. Such working units are detachably mounted on the operating unit. When the working units are selectively mounted on the operating unit, the pulleys in the proximal end of the working unit are held in engagement with the rotational shafts of the motors in the operating unit.

In a system where different working units are selectively connected to one operating unit, it is necessary to establish a motor phase, which serves as a common axis position for allowing all of the working units to be detachably mounted on the operating unit (see, for example, JP 2004-105451 A and JP 08-071072 A). The established motor phase is referred to as an origin or initial position.

The motors and the working unit are operatively coupled to each other by wires. Therefore, even when the motors are returned to their original states, the working unit may not fully be returned to its initial position, but may suffer from a positional error, due to inevitable stretching of the wires and friction between the various parts.

In order to prevent the working unit from suffering from positional errors, a sensor may be provided at a location corresponding to the working unit, rather than the motors, and the working unit may be controlled by a feedback loop so as to return the working unit to its initial position based on detected signals from the sensor (see, for example, JP 2002-261496 A and JP 2006-149468 A).

Medical manipulators should be designed based on certain desirable conditions owing to the fact that the working units thereof are inserted into body cavities. According to such desirable conditions, a medical manipulator should be as small and light as possible, should be mounted replaceably on an operating unit, should be able to be cleaned and sterilized easily, and should not include electrical devices therein, except for an electrosurgical knife or the like.

According to the inventions disclosed in JP 2002-261496 A and JP 2006-149468 A, for returning the working unit reliably to its initial position or origin, the sensor is provided at a location corresponding to the working unit. If the sensor is incorporated in a medical manipulator, then the working unit becomes large and heavy. Particularly, if the distal end of the working unit is unduly heavy, the working unit is subject to a large moment and cannot easily be operated. Also, the working unit incorporating the sensor cannot easily be replaced, because the sensor needs to be electrically connected. In addition, in such a case, the working unit becomes difficult to clean and sterilize.

### SUMMARY OF THE INVENTION

It is one of the objects of the present invention to provide a manipulator system and a method of controlling a manipulator for reliably returning a working unit to an origin or initial position, without the need for an electrical device such as a sensor or the like to be used in combination with the working unit.

According to one aspect of the present invention, a manipulator system is provided comprising a manipulator having an actuator and an actuating unit, the actuating unit being actuatable by the actuator to change an attitude thereof, and a controller for controlling the manipulator in order to perform an origin return process for moving the actuating unit to an end of an operating range thereof, by issuing a first control target value to the actuator indicative of a virtual position beyond the end of the operating range, and thereafter issuing a second control target value to the actuator indicative of the end of the operating range.

According to another aspect of the present invention, a method of controlling a manipulator also is provided having an actuating unit which can be actuated by an actuator in order to change an attitude thereof, comprising performing an origin return process for moving the actuating unit to an end of an operating range thereof, by issuing a first control target value to the actuator indicative of a virtual position beyond the end of the operating range, and thereafter issuing a second control target value to the actuator indicative of the end of the operating range.

When the first control target value indicative of the virtual position is issued, the actuating unit reliably reaches the end of its operating range. Therefore, the actuating unit can reliably be returned to its origin without the need for an electric device such as a sensor in combination with the actuating unit. At this time, stresses are not yet removed from the actuating unit, the actuator, and a transmitting member. Therefore, the second control target value, which indicates the end of the operating range, is subsequently issued in order to remove such stresses.

Even if the actuator and the actuating unit are operatively connected to each other by the transmitting member, which inevitably experiences stretching, and by various parts that cause friction, the actuating unit can accurately be returned to the origin thereof, thereby eliminating error.

The above and other objects, features, and advantages of the present invention will become more apparent from the following description when taken in conjunction with the accompanying drawings in which a preferred embodiment of the present invention is shown by way of illustrative example.

### BRIEF DESCRIPTION OF THE DRAWINGS

FIG. 1 is a perspective view of a manipulator system according to an embodiment of the present invention;

FIG. 2 is a perspective view showing possible combinations of the manipulator system according to the embodiment of the present invention;

FIG. 3 is a side elevational view of a manipulator of the manipulator system, with a working unit and an operating unit being separated from each other;

FIG. 4 is a perspective view of the operating unit;

FIG. 5 is a perspective view of a distal-end working unit of the working unit;

FIG. 6 is an exploded perspective view of the distal-end working unit;

FIG. 7 is a block diagram of a controller of the manipulator system;

FIG. 8 is a flowchart of a processing sequence of the manipulator system;

FIG. 9 is a flowchart of a sequence of an origin return process;

FIG. 10 is a graph showing control target values and changes in the angle of a gripper in the distal-end working unit during the origin return process;

FIG. 11 is a schematic perspective view showing a motor and the gripper in a first state in the origin return process;

FIG. 12 is a schematic perspective view showing the motor and the gripper in a second state in the origin return process;

FIG. 13 is a schematic perspective view showing the motor and the gripper at a time when the origin return process is ended; and

FIG. 14 is a schematic perspective view of a surgical robot system with the working unit connected to the distal end of a robot arm.

### DESCRIPTION OF THE PREFERRED EMBODIMENT

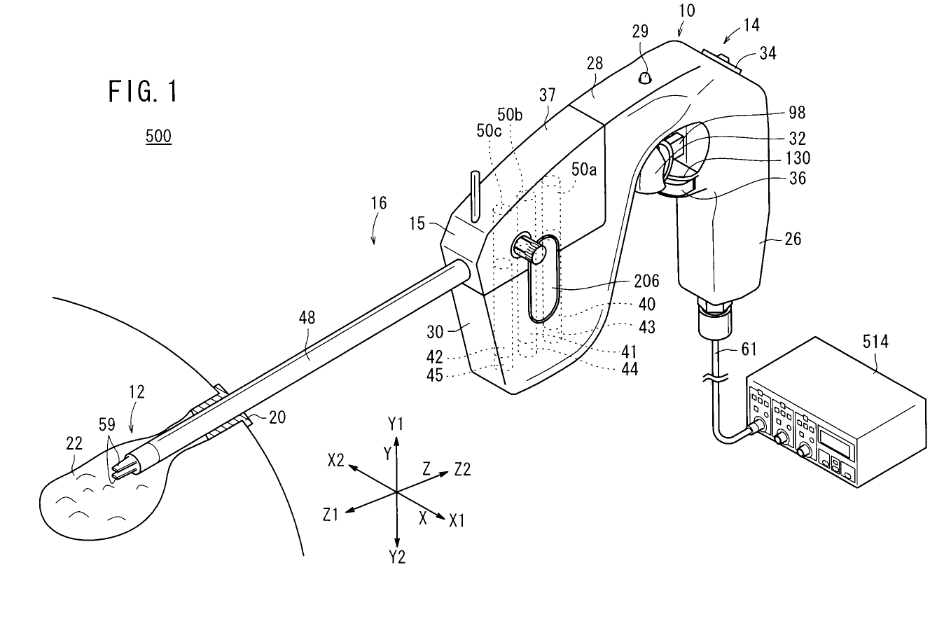

A manipulator system 500 according to an embodiment of the present invention will be described below with reference to FIGS. 1 through 14. The manipulator system 500 (see FIG. 1) may be used in a laparoscopic surgical operation process or the like.

As shown in FIG. 1, the manipulator system 500 comprises a manipulator 10 and a controller 514 for controlling the manipulator 10. The manipulator 10 and the controller 514 are detachably connected to each other by a connector.

The manipulator 10 includes a distal-end working unit 12 for gripping a portion of a living tissue, or for gripping a curved needle or the like, for performing a given surgical treatment. The manipulator 10 comprises an operating unit (first portion) 14 and a working unit (second portion) 16 as basic components. The controller 514 electrically controls the manipulator 10, and is connected by the connector to a cable 61 that extends from the lower end of a grip handle 26 of the operating unit 14.

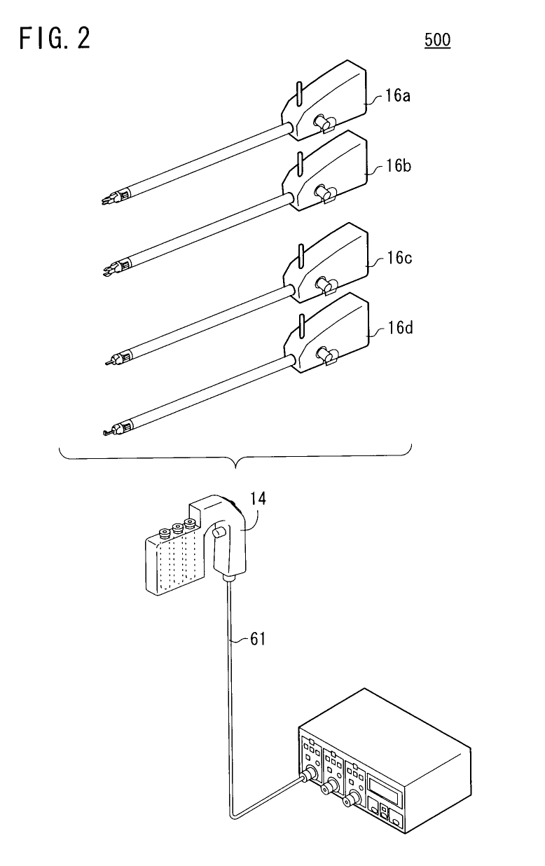

As shown in FIG. 2, the manipulator system 500 may have various configurations selectively. Specifically, working units 16a, 16b, 16c, 16d are available as variations for the working unit 16, and can selectively be mounted on the operating unit 14. The surgeon or operator who handles the manipulator system 500 can select one of the working units 16a, 16b, 16c, 16d, depending on the surgical procedure that the surgeon intends to perform, and the degree to which the surgeon is familiar with the working units. The working unit 16b has scissors as the distal-end working unit 12 thereof. The working unit 16c has a blade-like electrosurgical knife as the distal-end working unit 12 thereof. The working unit 16d has a hook-like electrosurgical knife as the distal-end working unit 12 thereof. The working units 16a, 16b, 16c, 16d have common pulleys 50a, 50b, 50c (see FIG. 1) disposed within the connectors 15 thereof.

The manipulator 10, which comprises the operating unit 14 and the working unit 16a, will be described below.

The distal-end working unit 12 of the manipulator 10 serves to grip a portion of a living tissue, a curved needle, or the like, for performing a given surgical treatment. The distal-end working unit 12 is usually referred to as a gripping forceps or a needle driver (needle holder).

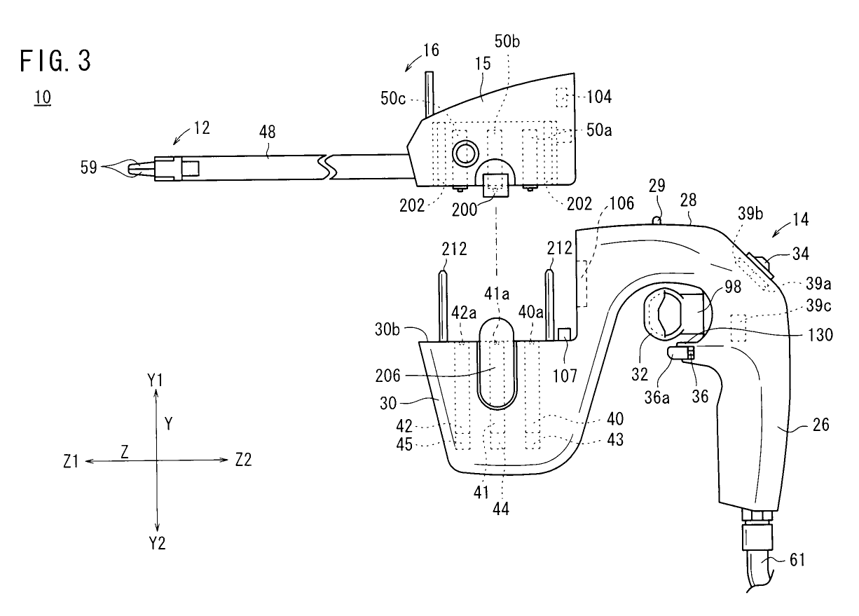

As shown in FIGS. 1 and 3, the manipulator 10 includes the operating unit 14, which is held and operated by hand, and the working unit 16, which is detachably mounted on the operating unit 14.

In the following descriptions, transverse directions in FIG. 1 shall be referred to as X directions, vertical directions as Y directions, and longitudinal directions of a hollow joint shaft 48 as Z directions. In the X direction, the rightward direction as viewed from the distal end is referred to as an X1 direction, and the leftward direction as an X2 direction. In the Y direction, the upward direction is referred to as a Y1 direction, and the downward direction as a Y2 direction. In the Z direction, the forward direction is referred to as a Z1 direction, and the rearward direction as a Z2 direction. Unless otherwise noted, these directions represent directions of the manipulator 10 when the manipulator is placed in a neutral attitude. The definitions of the above directions are for illustrative purposes only, and the manipulator 10 can be used in any orientation. For example, the manipulator 10 may be used upside down.

The working unit 16 includes the distal-end working unit 12 for performing a working operation, a connector 15 connected to an actuator block 30 of the operating unit 14, and an elongate hollow joint shaft 48 coupling the distal-end working unit 12 and the connector 15 to each other. When a predetermined action is performed on the actuator block 30, the working unit 16 can be separated from the operating unit 14, so that the working unit 16 can be cleaned, sterilized, and/or serviced for maintenance.

The distal-end working unit 12 and the joint shaft 48, which are small in diameter, can be inserted into a body cavity 22 through a trocar 20 in the form of a hollow cylinder mounted in an abdominal region or the like of the patient. The distal-end working unit 12 is actuated by the operating unit 14 in order to perform various surgical techniques to remove, grip, suture, or ligate an affected area of the patient's body inside the body cavity 22.

The operating unit 14 includes a grip handle 26 gripped by hand, a bridge 28 extending from an upper portion of the grip handle 26, and an actuator block 30 connected to a distal end of the bridge 28.

As shown in FIG. 1, the grip handle 26 of the operating unit 14 extends in the Y2 direction from the end of the bridge 28, and has a length suitable for being gripped by a human hand. The grip handle 26 has a trigger lever 32 as an input means, a composite input unit 34, and a switch 36.

The bridge 28 has an LED 29 in an easily visually recognizable position on an upper surface or a side surface thereof. The LED 29 is an indicator for indicating a controlled state of the manipulator 10. The LED 29 is of a size large enough to be easily visually recognizable by the operator, and yet is sufficiently small and light so as not to interfere with the operation of the manipulator 10. In the present embodiment, the LED 29 is located in a visually recognizable position substantially centrally on the upper surface of the bridge 28.

The cable 61 has an end connected to the lower end of the grip handle 26 and an opposite end connected to the controller 514. The grip handle 26 and the cable 61 may be connected to each other by a connector.

The composite input unit 34 serves as a composite input means for giving rotational commands in rolling directions (shaft rotating directions) and yawing directions (left and right directions) to the distal-end working unit 12. For example, the composite input unit 34 may serve as a first input means movable in the shaft rotating directions for entering commands in rolling directions, and a second input means movable in left and right directions for entering commands in yawing directions. The trigger lever 32 serves as an input means for giving opening and closing commands to a gripper (actuating unit) 59 (see FIG. 1) of the distal-end working unit 12. The controller 514 holds internal signals indicative of angles of the motors 40, 41, 42 corresponding to a roll axis, a yaw axis, and a gripper axis respectively. Based on signals from the composite input unit 34 and the trigger lever 32, the controller 514 changes the internal signals to equalize the angles of the motors 40, 41, 42.

The switch 36 serves as an input means for selectively enabling and disabling the manipulator 10.

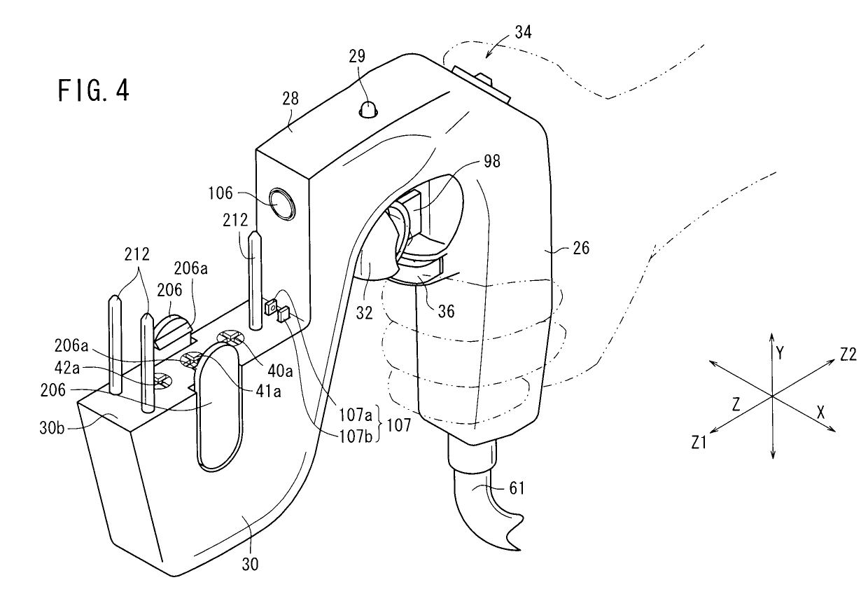

As shown in FIGS. 3 and 4, the composite input unit 34 and the trigger lever 32 are associated with input sensors 39a, 39b, 39c for detecting operational quantities. The input sensors 39a, 39b, 39c supply detected operational signals to the controller 514.

The trigger lever 32 is disposed slightly below the bridge 28 and projects in the Z1 direction. The trigger lever 32 is disposed in a position where it can easily be operated by the index finger of the hand that grips the grip handle 26.

The trigger lever 32 is operatively coupled to the grip handle 26 by an arm 98, and is movable toward and away from the grip handle 26.

The switch 36 serves as an operating mechanism movable toward and away from the grip handle 26. The trigger lever 32 and the switch 36 extend in the Z1 direction away from the grip handle 26 and are located closely together, i.e., juxtaposed in the longitudinal direction (Y direction) of the grip handle 26. The switch 36 is disposed directly below the trigger lever 32 in the Y2 direction, with a thin plate 130 interposed between the switch 36 and the trigger lever 32. The thin plate 130 extends from the grip handle 26 in the Z1 direction.

The switch 36 comprises an alternate switch having a trigger knob 36a. The switch 36 operates as follows: When the trigger knob 36a is pushed toward the grip handle 26 in the Z2 direction, the switch 36 is locked in an ON state, and the trigger knob 36a is held in a position near the grip handle 26. When the trigger knob 36a is pushed again toward the grip handle 26, the switch 36 is released from the ON state into an OFF state. The trigger knob 36a is automatically returned in the Z1 direction to a position remote from the grip handle 26 under the bias of a resilient member (not shown).

Operation and stop modes of the manipulator 10 are changed by the switch 36. Specifically, the controller 514, which reads the states of the switch 36, places the manipulator 10 in an operational mode when the switch 36 is in the ON state, operates the manipulator 10 under an automatic origin return process in order to return the motors 40, 41, 42 to the origin when the switch 36 changes from the ON state to the OFF state, and places the manipulator 10 in a stop mode after the motors 40, 41, 42 have been returned to their origin positions. The operational mode is a mode in which the distal-end working unit 12 is actuated based on operations of the trigger lever 32 and the composite input unit 34. The stop mode is a mode in which the distal-end working unit 12 is inactivated in a predetermined origin attitude.

The controller 514 distinguishes the above modes and processes from each other, and changes the energized state of the LED 29 based on the distinguished modes and processes.

The actuator block 30 houses therein the motors 40, 41, 42, which are associated respectively with mechanisms having three degrees of freedom in the distal-end working unit 12. The motors 40, 41, 42 are juxtaposed along the direction in which the joint shaft 48 extends. The motors 40, 41, 42 are small in size and diameter, and the actuator block 30, which houses the motors 40, 41, 42 therein, is of a flat compact shape. The actuator block 30 is disposed below an end of the operating unit 14 in the Z1 direction. The motors 40, 41, 42 are energized under the control of the controller 514 based on actions made by the operator on the operating unit 14.

The motors 40, 41, 42 are combined with respective angle sensors (detecting means) 43, 44, 45 for detecting respective angular displacements of the motors 40, 41, 42. The angle sensors 43, 44, 45 supply detected angle signals to the controller 514. The angle sensors 43, 44, 45 may comprise rotary encoders, for example.

The working unit 16 includes on its proximal end the connector 15 connected to the actuator block 30 and the hollow joint shaft 48 that extends from the connector 15 in the Z1 direction. The connector 15 houses pulleys (driven members) 50a, 50b, 50c rotatably disposed therein, which are connected respectively to rotatable shafts 40a, 41a, 42a of the motors 40, 41, 42. The pulleys 50a, 50b, 50c each have respective couplings.

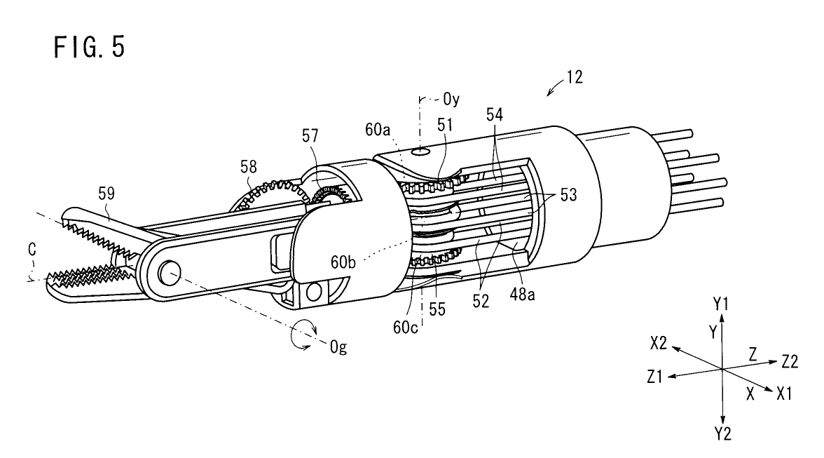

Wires (transmitting members) 52, 53, 54 are trained around the pulleys 50a, 50b, 50c, respectively, and extend through a space 48a (see FIG. 5) in the hollow joint shaft 48 to the distal-end working unit 12. The wires 52, 53, 54 may be of the same type and diameter.

The working unit 16 can be separated from the operating unit 14 by carrying out a given action on the actuator block 30, so that the working unit 16 can be cleaned, sterilized, or serviced for maintenance. The working unit 16 may be replaced with another type of working unit (see FIG. 2). Depending on the nature of the surgical procedure to be carried out using the manipulator 10, a working unit 16 whose joint shaft 48 has a different length or whose distal-end working unit 12 has a different mechanism may be mounted on the operating unit 14.

The working unit 16 is detachably mounted on the operating unit 14. When the working unit 16 is mounted on the operating unit 14, the rotatable shafts 40a, 41a, 42a of the motors 40, 41, 42 are held in axially aligned engagement with the lower ends of the pulleys 50a, 50b, 50c. Specifically, the lower ends of the pulleys 50a, 50b, 50c have respective criss-crossed joint teeth thereon, and the upper ends of the rotatable shafts 40a, 41a, 42a have respective criss-crossed joint recesses defined therein. When the working unit 16 is mounted on the operating unit 14, the criss-crossed joint teeth on the lower ends of the pulleys 50a, 50b, 50c are fitted into the respective joint recesses provided in the upper ends of the rotatable shafts 40a, 41a, 42a, for thereby reliably transferring rotational forces from the motors 40, 41, 42 to the pulleys 50a, 50b, 50c. The joint teeth and the joint recesses are not limited to criss-crossed shapes, but may comprise other types of interfitting shapes as well.

The connector 15 has an ID (identification) holder 104 for holding an ID capable of individually identifying the working unit 16.

The ID holder 104 may be a wireless ID holder such as an RFID (Radio Frequency Identification) holder, a non-contact detection ID holder such as an optical ID holder, which may be a bar code, a matrix two-dimensional code, or the like, or a contact detection ID holder such as a sequence of small protrusions or the like.

The ID held by the ID holder 104 includes a value for identifying each of the working units 16a through 16d.

The ID holder 104 does not need to be electrically energized directly, and hence the connector 15 and the working unit 16 have no electric contacts. Therefore, when the working unit 16 is dismounted from the operating unit 14, the working unit 16 can easily be cleaned or sterilized. Specifically, all of the electric components, including the motors, the switches, and the sensors, are placed within the operating unit 14, whereas only mechanical components including the joint shaft 48 and the distal-end working unit 12 are provided in the working unit 16, so that the working unit 16 can efficiently be cleaned. It is preferable for the working unit 16 and the operating unit 14 to be separable from each other, since under use the units will be smeared differently with different materials, thus requiring the units to be cleaned and serviced for maintenance using different techniques. Since no electric components are included in the working unit 16, the working unit 16 can easily be replaced on the operating unit 14.

Since the distal-end working unit 12 is free of electric components, it is small in size, small in diameter, and light in weight. Furthermore, since the weight at the distal end of the distal-end working unit 12 is small, the distal-end working unit 12 is subject to a small moment, thus allowing the manipulator 10 to be operated with ease.

The operating unit 14 has an ID relay unit (identifying means) 106 for reading the ID held by the ID holder 104 and supplying the read ID to the controller 514. The ID relay unit 106 may comprise an RFID transmitting and receiving circuit, a photocoupler, or the like.

The actuator block 30 has a pair of levers 206 pivotally mounted on respective outer side surfaces thereof. The levers 206 have respective wedges 206a on upper inner surfaces thereof, which engage respective teeth 200 on outer side surfaces of the connector 15 when the connector 15 is mounted on the actuator block 30. The levers 206 are normally biased by a resilient member to hold the wedges 206a in locking engagement with the teeth 200. For removing the connector 15 from the operating unit 14, the operator pushes the lower portions of the levers 206 in order to tilt the upper portions thereof outwardly, thereby releasing the edges 206a from engagement with the teeth 200. The connector 15 can now be pulled upwardly in the Y1 direction and detached from the operating unit 14. The actuator block 30 has three alignment pins 212 that project upwardly from the upper surface thereof. The connector 15 has three fitting holes 202 defined therein, which open in a downward direction. When the alignment pins 212 are fitted respectively in the fitting holes 202, the connector 15 is stably supported on the actuator block 30. For installing the connector 15 on the operating unit 14, the alignment pins 212 are positioned in alignment with the respective fitting holes 202, and then the connector 15 is pressed downwardly in the Y2 direction toward the actuator block 30. As the connector 15 is displaced toward the actuator block 30, the upper portions of the levers 206 are spread outwardly by the teeth 200. When the wedges 206a move past the teeth 200, the levers 206 snap back under the resiliency of the resilient member, thereby bringing the wedges 206a into locking engagement with the teeth 200, so that the connector 15 becomes locked in place on the actuator block 30.

A working unit detecting means 107 for detecting whether the connector 15 has been placed on the actuator block 30 is disposed on an upper surface 30b of the actuator block 30, at an end thereof in the Z2 direction. The working unit detecting means 107 comprises a light emitter 107a and a light detector 107b, which are positioned in confronting relation to each other. When a portion of the rear end of the connector 15 is inserted between the light emitter 107a and the light detector 107b, the connector 15 blocks light emitted from the light emitter 107a toward the light detector 107b, thereby detecting that the connector 15 has been mounted on the actuator block 30. The light emitter 107a and the light detector 107b confront each other in the X direction and are disposed closely to each other. The light emitter 107a may be an LED, and the light detector 107b may be a photodiode, for example.

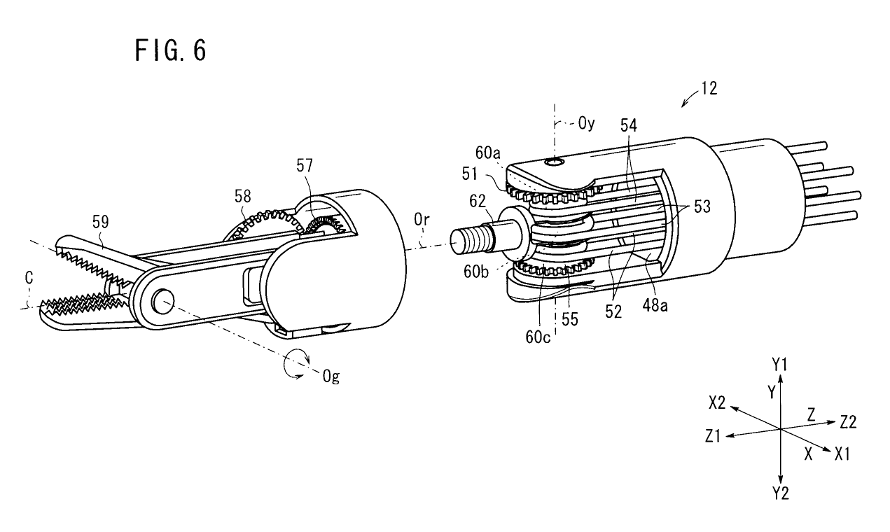

As shown in FIGS. 5 and 6, the distal-end working unit 12 incorporates therein mechanisms providing three degrees of freedom. These mechanisms include a mechanism (tilting mechanism, pivot axis) having a first degree of freedom for angularly moving a distal end portion, which is positioned in front of a first rotational axis Oy extending along the Y direction, in yawing directions about the first rotational axis Oy, a mechanism (rolling mechanism) having a second degree of freedom for angularly moving the distal end portion in rolling directions about a second rotational axis Or extending along the Z direction, and a mechanism having a third degree of freedom for opening and closing the gripper 59 on the distal end about a third rotational axis Og extending along the X direction.

The first rotational axis Oy of the mechanism having the first degree of freedom may be an axis about which the distal end portion is angularly movable, and not parallel to an axis C that extends from the proximal to the distal end of the joint shaft 48. The second rotational axis Or of the mechanism having the second degree of freedom may be an axis along which the distal end (the gripper 59) of the distal-end working unit 12 extends and about which the gripper 59 is rotatable in the rolling directions.

The gripper 59 is fully closed when in the origin position and can be opened through a given angle from the origin. Although the gripper 59 is shown as being a one-sided openable type, the gripper 59 may also be a double-sided openable type. A one-sided openable type refers to a structure in which one of a pair of pinching members of the gripper 59 is openable and closable with respect to the other pinching member, which is fixed. A double-sided openable type refers to a structure in which both of the pinching members of the gripper 59 are openable and closable with respect to each other.

The distal-end working unit 12 is actuated by the wires 52, 53, 54, which are trained around corresponding tubular members 60c, 60b, 60a that are supported rotatably in the distal end of the joint shaft 48.

When the wires 52, 54 are moved, gears 51, 55 coaxial with the tubular members 60c, 60b, 60a are rotated, thereby causing a face gear, not shown, to rotate the gripper 59 in the rolling directions. When the wire 54 is moved, the gear 51 is rotated, thereby causing a face gear 57 and a gear 58 to open and close the gripper 59. When the wires 52, 53, 54 are moved, a main shaft 62 is angularly moved in order to turn the gripper 59 in the yawing directions.

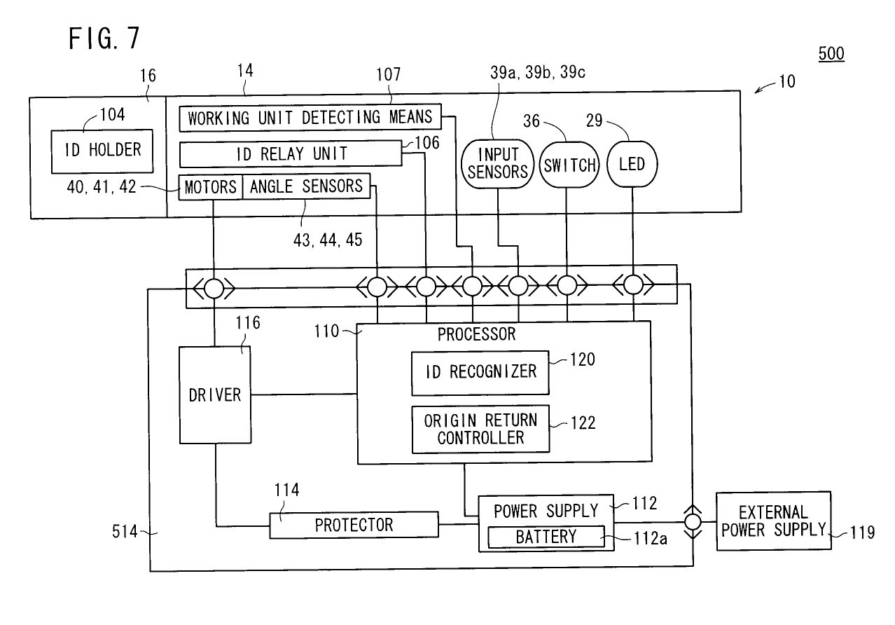

Internal structural details of the controller 514 will be described below with reference to FIG. 7.

As shown in FIG. 7, the controller 514 includes a processor 110, a power supply 112, a protector 114, and a driver 116. The power supply 112 regulates electric power supplied from an external power supply 119 and supplies the regulated electric power to various components in the controller 514. The power supply 112 charges a battery 112a, and automatically switches to the battery 112a in the event that electrical power cannot be supplied from the external power supply 119. The power supply 112 thus operates as an uninterruptible power supply. The battery 112a is connected in parallel with a transformer-rectifier assembly in the power supply 112.

The protector 114 shuts off electric power supplied to the manipulator 10 based on various items of information supplied thereto. When the protector 114 shuts off the electric power supplied to the manipulator 10, the manipulator 10 immediately stops operation.

The processor 110 is electrically connected to the angle sensors 43, 44, 45, the input sensors 39a, 39b, 39c, and the switch 36. Based on signals from these sensors and the switch 36, the processor 110 determines how to operate the manipulator 10, supplies a predetermined command signal to the driver 116, and controls an operational state display unit to display a certain operational state. The processor 110 also is connected electrically to the LED 29 in order to control the energized state thereof. The processor 110 is further electrically connected to various switches on the front panel of the controller 514 for controlling the switches. The processor 110 comprises a CPU, a ROM, a RAM, etc., and performs given software processes by reading and executing a program.

The driver 116 is electrically connected to the motors 40, 41, 42, and energizes the motors 40, 41, 42 based on commands from the processor 110. A drive system for the motors 40, 41, 42 determines operational angle command values for the distal-end working unit 12 based on signals from the input sensors 39a, 39b, 39c, determines differences between the operational angle command values and the angle signals from the angle sensors 43, 44, 45, performs a predetermined compensating process based on such differences, and supplies command signals to the driver 116.

Therefore, the drive system for the motors 40, 41, 42 is of a closed loop configuration.

The processor 110 includes an ID recognizer (identifying means) 120 and an origin return controller 122. The ID recognizer 120 recognizes the ID of the ID holder 104.

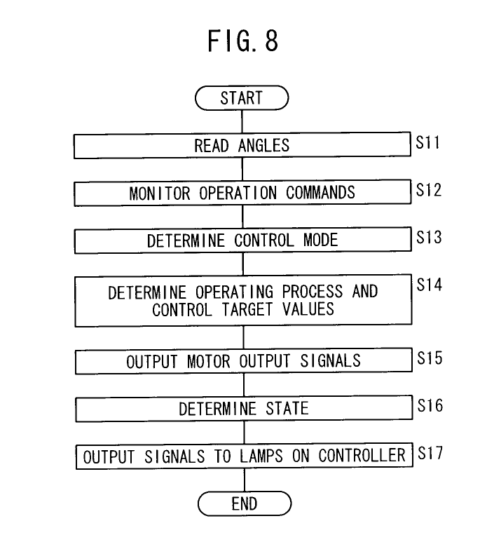

Operation of the manipulator system 500 will be described below with reference to the flowchart shown in FIG. 8.

The manipulator system 500 operates under the general control of the processor 110 of the controller 514, and basically performs an operation sequence according to the flowchart shown in FIG. 8. The operation sequence according to the flowchart shown in FIG. 8 is repeatedly carried out during predetermined control periods. It is assumed that the operation sequence is performed in the order of the indicated step numbers, unless otherwise indicated.

In step S11 shown in FIG. 8, the processor 110 reads output signals from angle detectors in the operating unit 14 and the angle sensors 43, 44, 45 of the motors 40, 41, 42.

In step S12, the processor 110 recognizes input signals from the command input means and the switch 36.

In step S13, the processor 110 determines a control mode for the manipulator 10 based on the input signals recognized by the processor 110.

In step S14, the processor 110 determines an operating process and control target values for the motors 40, 41, 42 according to the determined control mode.

In step S15, the processor 110 calculates motor output signals from the control target values and the angle signals from the angle sensors 43, 44, 45 according to a control process such as a PID control process, and outputs the calculated motor output signals to the driver 116.

In step S16, the processor 110 compares various defined conditions with the angle signals from the angle sensors 43, 44, 45, and determines the state of the manipulator 10.

In step S17, the processor 110 outputs signals to the lamps on the controller 514, based on the determined state of the manipulator 10.

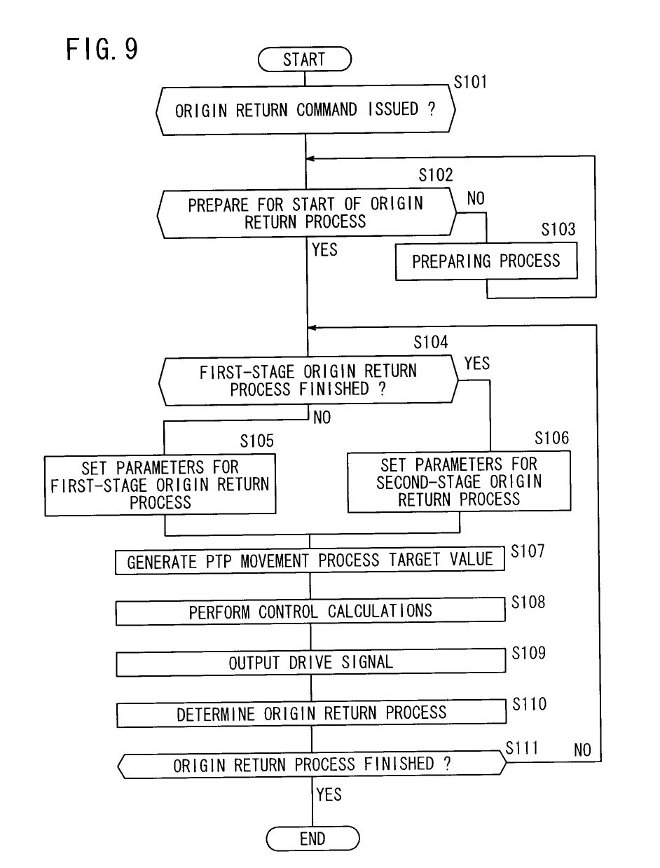

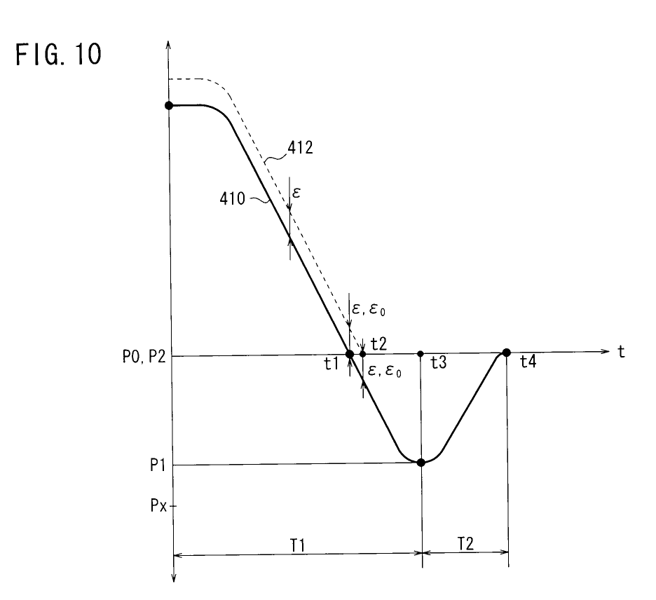

The origin return process will be described below with reference to FIGS. 9 through 13. The origin return process is performed by the controller 514 based on actions made on the switch 36 and the switches on the controller 514, and the origin return process is divided into a first-stage origin return process during an interval T1 (see FIG. 10) and a second-stage origin return process during an interval T2. With reference to FIG. 9, a process for automatically returning the gripper 59 to an end of the operating range thereof, i.e., to an origin P0 (see FIG. 10), will mainly be described below. Since the motor 40 is feedback-controlled based on the angle signal from the angle sensor 43, at least in a static state, it can be assumed that the angular displacement of the motor 40 is substantially free of positional errors from the control target value.

In step S101 shown in FIG. 9, the controller 514 monitors the state of the switch 36 in order to confirm whether an origin return command has been generated or not. If an origin return command has been generated by the switch 36, then control goes to step S102. If an origin return command has not been generated by the switch 36, then the controller 514 waits for an origin return command to be generated.

In step S102, the controller 514 determines whether or not preparations for starting the origin return process have been made. If preparations for starting the origin return process have been made, then control goes to step S104. If preparations for starting the origin return process have not been made, then the controller 514 performs a given preparatory process, in step S103, and then waits until it is determined again whether preparations for the origin return process have been started.

The preparations for starting the origin return process refer to conditions indicating that a predetermined servo flag is ON, that a first-stage origin return process flag and a second-stage origin return process flag are OFF, that a second-stage origin return process completion flag is OFF, and that the connected working unit 16 corresponds to a given type. The servo flag is a flag indicating that the motors 40, 41, 42 can be servo-controlled. The first-stage origin return process flag and the second-stage origin return process flag are flags indicating that the first-stage origin return process during the interval T1 and the second-stage origin return process during the interval T2 are being carried out. A first-stage origin return process completion flag and a second-stage origin return process completion flag are flags indicating that the first-stage origin return process during the interval T1 and the second-stage origin return process during the interval T2 have finished. The flags make the corresponding indications thereof affirmative when the flags are ON, and negative when the flags are OFF.

In step S104, the controller 514 determines whether the first-stage origin return process has finished or not. Specifically, the controller 514 monitors the first-stage origin return process completion flag in step S104. If the first-stage origin return process completion flag is ON, then control goes to step S106. If the first-stage origin return process completion flag is OFF, then control goes to step S105.

In step S105, the controller 514 sets given parameters for performing the first-stage origin return process. Specifically, the controller 514 acquires a present process start angle, establishes a first control target value P1, and turns the first-stage origin return process flag ON.

As shown in FIG. 10, the first control target value P1 is established as a virtual position beyond the origin P0 in the closing direction. The first control target value P1 represents a position, which is over from the origin P0 by a distance much greater than a value corresponding to an error ε between the target position for the gripper 59 and the actual position thereof, for thereby eliminating an error at the origin P0. The solid-line curve 410 shown in FIG. 10 represents the control target value and the angle of the motor 40 or the pulley 50a, whereas the broken-line curve 412 represents the actual opening of the gripper 59. The difference between the solid-line curve 410 and the broken-line curve 412 represents the error ε. If the error ε is not constant, then the first control target value P1 may be established based on an error ε₀ at the time the control target value is equal to the origin P0. In other words, the first control target value P1 may be established as P1<P0−ε₀. The first control target value P1 is also established as a position smaller than a limit value Px for the control target value for the gripper 59, at the time that the manipulator 10 operates normally other than during the origin return process. Stated otherwise, in order to cause the gripper 59 to generate forces for reliably gripping an object while the manipulator 10 operates normally, the control target value is set to a value within a range between the origin P0 and the limit value Px. The first control target value P1, which thus is established as a position smaller than the limit value Px, is prevented from becoming excessively large and makes it possible to reduce the time required to perform the origin return process.

In step S106, the controller 514 sets given parameters for performing the second-stage origin return process. Specifically, the controller 514 acquires a present process start angle at that time (which essentially is identical to the first control target value P1), establishes a second control target value P2, and turns the second-stage origin return process flag ON. The second control target value P2 is in agreement with the origin P0. After step S105 or S106, control goes to step S107.

In step S107, the controller 514 generates a target value for a PTP (Point-to-Point) movement process. The PTP movement process is a process for moving the gripper 59 along a target trajectory, which is generated to interconnect the present position and the target position. The PTP movement process is realized by linear interpolation, trapezoidal speed interpolation, or an S-shaped acceleration/deceleration trajectory between the present position and the target position.

In step S108, the controller 514 performs control calculations for performing the PTP movement process. Processing from steps S105 through S108 may be carried out only initially during the first-stage origin return process and during the second-stage origin return process.

In step S109, the controller 514 outputs a drive signal to the motor 40 based on the result of the control calculations.

In step S110, the controller 514 determines the origin return process. Specifically, during the first-stage origin return process, the controller 514 monitors whether or not the detected value from the angle sensor 43 has reached the first control target value P1. When the detected value from the angle sensor 43 reaches the first control target value P1, the controller 514 turns off the first-stage origin return process flag, while turning on the first origin return process completion flag.

During the second-stage origin return process, the controller 514 monitors whether or not the detected value from the angle sensor 43 has reached the second control target value P2 (=P0). When the detected value from the angle sensor 43 has reached the second control target value P2, the controller 514 turns off the second-stage origin return process flag, while turning on the second origin return process completion flag. The controller 514 may determine the origin return process with a given latitude in view of errors.

In step S111, the controller 514 determines whether or not the origin return process has finished. Specifically, if both the first origin return process completion flag and the second origin return process completion flag are ON, then the controller 514 judges that the origin return process has finished, and terminates the sequence shown in FIG. 9. Otherwise, control goes back to step S104.

If the control target value at the start of the origin return process is equal to the first control target value P1, then the first-stage origin return process is finished immediately, and the origin return process starts essentially from the second-stage origin return process.

If the control target value at the start of the origin return process is a value between the first control target value P1 and the limit value Px (i.e., if the gripper 59 is to be closed forcibly), then the controller 514 may turn the first-stage origin return process completion flag ON between step S102 and step S104, thereby simplifying the origin return process.

If a working unit is mounted on the operating unit 14, which does not need the first-stage origin return process during the interval T1 and the second-stage origin return process during the interval T2, e.g., a working unit 16c having a blade-like electrosurgical knife, then the controller 514 may turn the first-stage origin return process completion flag ON between step S102 and step S104, for performing an ordinary origin return process.

If the condition indicating that the second-stage origin return process completion flag is OFF is omitted from the conditions in step S102, then the origin control process can be carried out again. Such processing may be performed based on certain actions of the operator, when the origin control process experiences trouble due to an unexpected incident. Step S101 shown in FIG. 9 corresponds to step S12 shown in FIG. 8, steps S103 through S106 correspond to step S13, steps S107 and S108 correspond to step S14, and steps S109 through S110 correspond to step S15, respectively.

Operation of the manipulator 10 according to the above origin return process will be described below.

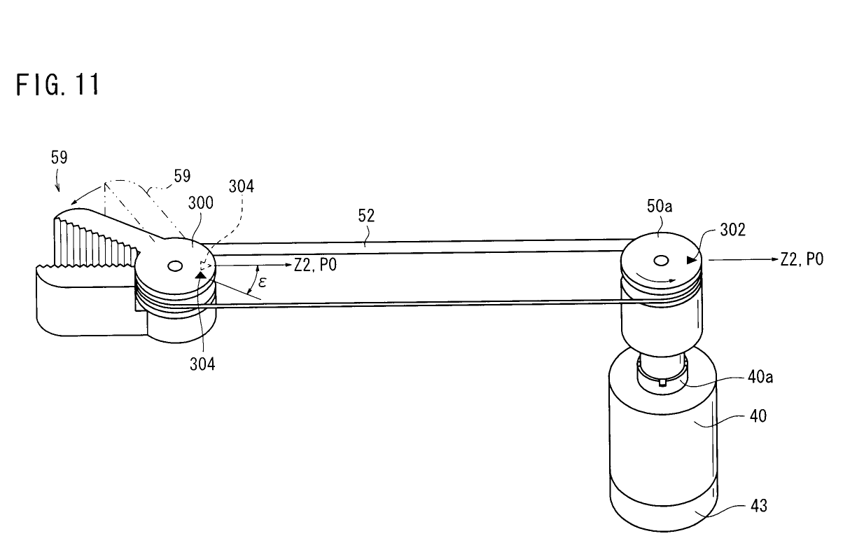

When the origin return process is started, the gripper 59 is open, as indicated by the imaginary lines in FIG. 11. In FIGS. 11 through 13, it is assumed that the speed reduction ratio between the motor 40, the pulley 50a and a rotor 300 of the gripper 59 is 1. Mechanisms with respect to the yaw and roll axes have been omitted from illustration. The mechanism for opening and closing the gripper 59 is shown in a simplified form. The pulley 50a and the rotor 300 are marked with respective markers 302, 304 for more easily understanding the angles thereof. The pulley 50a and the rotor 300 are placed in their origin positions P0 when the markers 302, 304 are oriented in the Z2 direction.

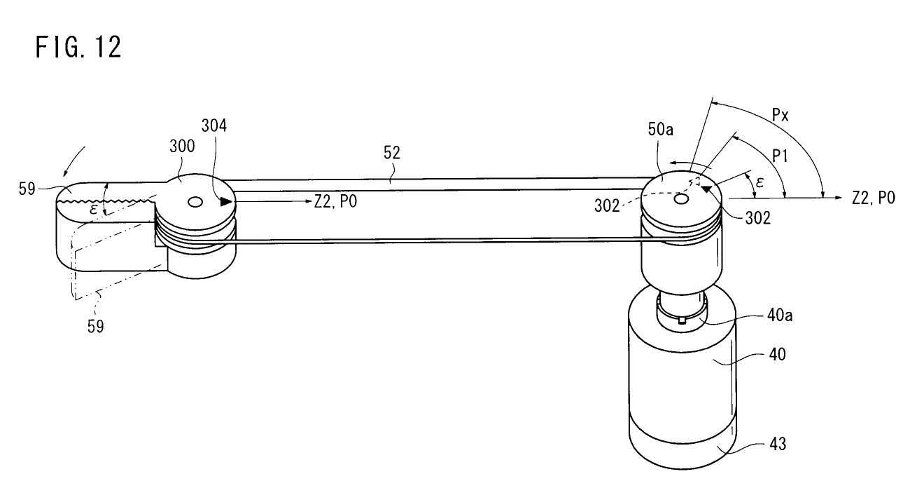

After the start of the origin return process, when the rotor 300 and the pulley 50a are turned counterclockwise in a direction to close the gripper 59 until time t1 (see FIG. 10) is reached, the pulley 50a reaches the origin P0. At this time, the rotor 300 does not reach the origin P0 but suffers from an error ε due to stretching of the wire 52 as well as friction between the various parts. In FIGS. 11 and 12, a portion of the wire 52, which is elongated under strong tension, is shown as being thinner, whereas a portion of the wire 52, which is held under a weak tension, is shown as being thicker.

If the error ε is not removed, then the handling of the manipulator 10 tends to feel odd or unusual. If the error ε is unduly large, then the operator finds it difficult to insert the distal-end working unit 12 through the trocar 20 (see FIG. 1). If the operator inactivates the manipulator 10 and disconnects the motor 40 and the pulley 50a from each other, then the wire 52 is restored to an unstretched state, thus causing the pulley 50a to turn clockwise.

According to the present invention, as shown in FIG. 12, the rotor 300 and the pulley 50a are turned further counterclockwise. At time t2 (see FIG. 10), the pulley 50a is turned beyond the origin P0 through an angle corresponding to the error ε, and the rotor 300 reaches the origin P0, thereby fully closing the gripper 59. At this time, the control command of the controller 514 represents a virtual position, which is angularly spaced from the closed position of the gripper 59, by an angle corresponding to the error ε (see the gripper 59 shown in imaginary lines in FIG. 12).

Although the gripper 59 is now closed, the pulley 50a is further turned counterclockwise to reach the first control target value P1, in order to fully close the gripper 59 in view of various control errors (see the marker 302 shown in imaginary lines in FIG. 12).

The gripper 59 is now fully closed. Since the first control target value P1 is smaller than the limit value Px, the wire 52 and the gripper 59 are prevented from being subjected to excessive forces, and the time required to perform the origin return process is shortened.

At this time, when the motor 40 and the pulley 50a are disconnected from each other, the wire 52 is restored to its unstretched state. However, since the wire 52 still undergoes a considerable amount of tension, the service life of the wire 52 is shortened. Furthermore, inasmuch as both the motor 40 and the pulley 50a are not in their origins P0, the manipulator system 500 may suffer from trouble, because replacement of the working unit 16 assumes that the motor 40 and the pulley 50a are in their origins P0. Specifically, it is physically difficult to remove the working unit 16 from the operating unit 14 and to mount another working unit 16 on the operating unit 14. Further, the software process for initializing the parameters based on the origin P0 becomes complex.

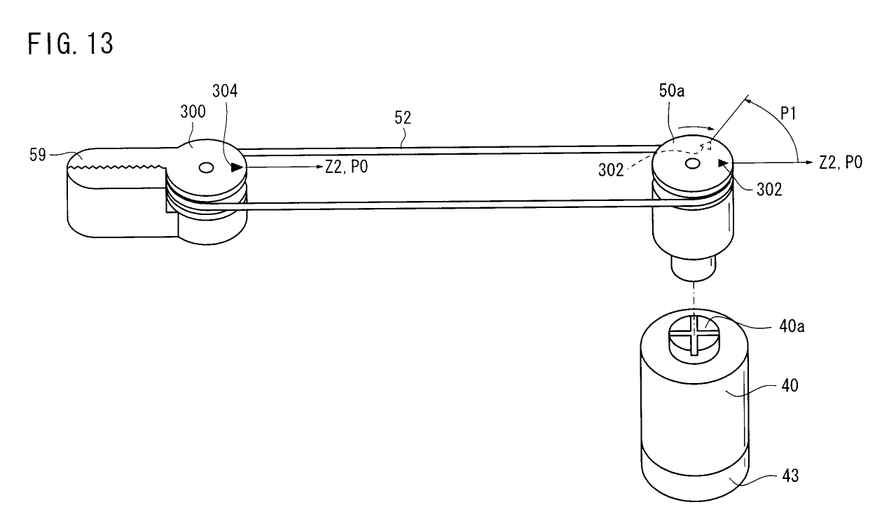

According to the present invention, as shown in FIG. 13, the pulley 50a and the rotor 300 are turned clockwise in a direction to open the gripper 59, until the pulley 50a and the rotor 300 reach their origins P0. At this time, the rotor 300 is not actually turned, but remains at the origin P0, while the wire 52 is restored to an unstretched state. Therefore, the motor 40, the pulley 50a, and the rotor 300 are stably held at their origin positions P0. When the motor 40 and the pulley 50a are disconnected from each other, since the tension of the wire 52 is essentially nil, or at its initial level, the rotor 300 and the pulley 50a are not moved. Therefore, the service life of the wire 52 can be increased.

According to the present embodiment, as described above, the first control target value P1 indicative of the virtual position is issued to cause the gripper 59 to reach the origin P0 at an end of the operating range thereof. Therefore, the gripper or the actuating unit 59 can reliably be returned to the origin without the need for an electric device, such as a sensor in combination with the gripper 59. At this time, since stresses remain and are not removed from the gripper 59, the motor 40, and the wire 52, the second control target value P2 indicative of the origin P0 is subsequently issued in order to remove such stresses.

Although the gripper 59 and the pulley 50a are operatively connected to each other by the wire 52, which inevitably experiences stretching, and by various parts of the manipulator which cause friction, the gripper 59 can accurately be returned to its origin position, thereby eliminating the error ε.

Since the origin P0 of the gripper 59 is the closed position of the gripper 59, the operator can visually confirm with ease the return of the gripper 59 to its origin. The operator thus finds it easy to insert the fully closed gripper 59 through the trocar 20.

The motor 40 is associated with the angle sensor 43 for detecting an angular displacement thereof. During the first-stage origin return process, the controller 514 monitors the detected value from the angle sensor 43. After having confirmed that the motor 40 has reached the first control target value P1, the controller 514 starts the second-stage origin return process. Since the motor 40 reliably reaches the first control target value P1, the error ε of the gripper 59 is reliably eliminated.

Since no electric components are included in the working unit 16, the working unit 16 that has been removed from the operating unit 14 can easily be cleaned and sterilized.

The controller 514 may change the first control target value P1 depending on the type of working unit 16, which is provided by the ID relay unit 106. Therefore, the controller 514 can appropriately control the working unit 16 based on the type of working unit 16.

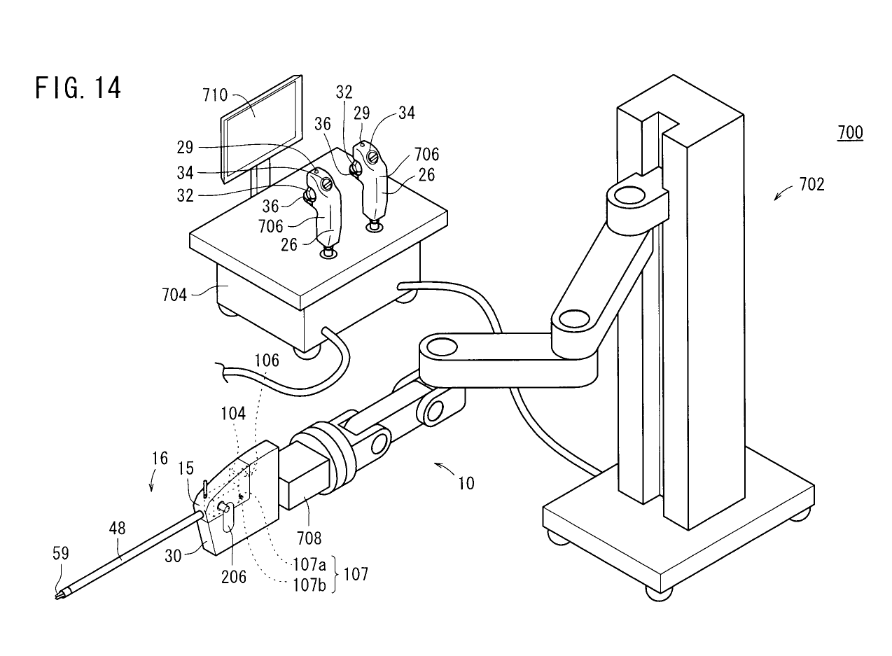

The working unit 16 has been described as being connected to a manually operable operating unit 14. However, the working unit 16 may also be applied to a surgical robot system 700, as shown in FIG. 14, for example.

The surgical robot system 700 has an articulated robot arm 702 and a console 704, with the working unit 16 being connected to the distal end of the robot arm 702. The distal end of the robot arm 702 incorporates a mechanism therein, which is the same as the actuator block 30, for connecting and actuating the working unit 16. The manipulator 10 comprises the robot arm 702 and the working unit 16. The robot arm 702 may comprise a means for moving the working unit 16, and is not limited to an installed type, but may be of an autonomous movable type. The console 704 may be a table type console, a control panel type console, or the like.

The robot arm 702 should preferably have six or more independent joints (rotary shafts, slide shafts, etc.) for setting the position and orientation of the working unit 16 as desired. The actuator block 30 on the distal end of the robot arm 702 is integrally combined with a distal end 708 of the robot arm 702.

The robot arm 702 operates under the control of the console 704, and may be actuated automatically according to a program, or by joysticks (robot operating units) 706 mounted on the console 704, or by a combination of the program and the joysticks 706. The console 704 includes and carries out the functions of the controller 514.

The console 704 includes two joysticks 706 as an operating unit, exclusive of the actuator block 30 of the above operating unit 14, and a monitor 710. Although not shown, the two joysticks 706 are capable of individually operating two robot arms 702. The two joysticks 706 are disposed in respective positions where they can easily be operated by both hands of the operator. The motor 710 displays information, such as an image produced by an endoscope.

The joysticks 706 can be moved vertically and horizontally, twisted, and tilted, and the robot arm 702 can be moved depending on such movements of the joysticks 706.

The joysticks 706 may be master arms. The robot arm 702 and the console 704 may communicate with each other via a communication means comprising a wired link, a wireless link, a network, or a combination thereof.

The manipulator system 500 according to the present invention is not limited to medical use, but also is applicable to other fields, including the repair of various parts in small spaces utilized in energy systems or the like.

Although a certain preferred embodiment of the present invention has been shown and described in detail, it should be understood that various changes and modifications may be made to the embodiment without departing from the scope of the invention as set forth in the appended claims.

What is claimed is:

1. A manipulator system comprising:
a manipulator having an actuator and an actuating unit, the actuating unit being actuatable by the actuator to change an attitude thereof; and
a controller for controlling the manipulator in order to perform an origin return process for moving the actuating unit to an end of an operating range thereof, by issuing a first control target value to the actuator indicative of a virtual position beyond the end of the operating range, and thereafter issuing a second control target value to the actuator indicative of the end of the operating range.

2. A manipulator system according to claim 1, wherein the actuating unit comprises an openable and closable gripper mounted on a distal end of the manipulator, and the end represents a closed position of the gripper.

3. A manipulator system according to claim 2, wherein the virtual position represents a position over from the end by a distance, which is greater than a value corresponding to an error between a target position of the actuating unit and an actual position thereof.

4. A manipulator system according to claim 2, wherein the virtual position represents a position, which is smaller than a limit value for a control target value for the gripper, when the gripper operates in a normal process other than the origin return process.

5. A manipulator system according to claim 1, wherein the actuator includes detecting means for detecting an operated position thereof, and the controller monitors a detected value from the detecting means, confirms that the actuator has reached the first control target value after having issued the first control target value, and then issues the second control target value.

6. A manipulator system according to claim 1, wherein the manipulator comprises:
a first portion including the actuator and being connected to the controller; and
a second portion having the actuating unit on a distal end thereof and a proximal end detachably connected to the first portion;
the second portion including a driven member disposed in the proximal end thereof and engaging the actuator, and a transmitting member for transmitting power from the driven member to the actuating unit.

7. A manipulator system according to claim 6, wherein the manipulator comprises a plurality of second portions each having an identifier indicative of the type thereof;
the first portion has identifying means for identifying the identifier of one of the second portions, which is mounted on the first portion, and supplying the type indicated by the identified identifier to the controller; and
the controller changes the virtual position depending on the type of the second portion that is supplied from the identifying means.

8. A method of controlling a manipulator having an actuating unit, which is actuatable by an actuator to change an attitude thereof, comprising:
performing an origin return process for moving the actuating unit to an end of an operating range thereof, by issuing a first control target value to the actuator indicative of a virtual position beyond the end of the operating range; and
thereafter issuing a second control target value to the actuator indicative of the end of the operating range.

9. A method according to claim 8, wherein the actuating unit comprises an openable and closable gripper mounted on a distal end of the manipulator, and the end represents a closed position of the gripper.

10. A method according to claim 9, wherein the virtual position represents a position over from the end by a distance, which is greater than a value corresponding to an error between a target position of the actuating unit and an actual position thereof.

11. A method according to claim 10, wherein the virtual position represents a position, which is smaller than a limit value for a control target value for the gripper, when the gripper operates in a normal process other than the origin return process.

12. A method according to claim 8, wherein the actuator includes detecting means for detecting an operated position thereof, and the controller monitors a detected value from the detecting means, confirms that the actuator has reached the first control target value after having issued the first control target value, and then issues the second control target value.

13. A method according to claim 8, wherein the manipulator comprises:
a first portion including the actuator and being connected to the controller; and
a second portion having the actuating unit on a distal end thereof and a proximal end detachably connected to the first portion;
the second portion including a driven member disposed in the proximal end thereof and engaging the actuator, and a transmitting member for transmitting movement from the driven member to the actuating unit.

14. A method according to claim 13, wherein the manipulator comprises a plurality of second portions each having an identifier indicative of the type thereof;
the first portion has identifying means for identifying the identifier of one of the second portions, which is mounted on the first portion, and supplying the type indicated by the identified identifier to the controller; and
the controller changes the virtual position depending on the type of the second portion that is supplied from the identifying means.
